(19) United States
(12) Patent Application Publication
(10) Pub. No.: US 2007/0123769 A1
(43) Pub. Date: May 31, 2007

(54) Tonometer-pachymeter apparatus for measurement of intraocular pressure

(76) Inventors: Terry A. Fuller, Jenkintown, PA (US); Francis E. O'Donnell JR., Town and Country, MO (US); Yongping Wang, Philadelphia, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/542,761

(22) Filed: Oct. 4, 2006

Related U.S. Application Data
(63) Continuation-in-part of application No. 10/890,615, filed on Jul. 14, 2004, now abandoned.
(60) Provisional application No. 60/724,086, filed on Oct. 6, 2005.
(60) Provisional application No. 60/489,681, filed on Jul. 24, 2003.

(51) Int. Cl. A61B 3/16; A61B 8/00

(52) U.S. Cl. 600/405; 600/449

(57) ABSTRACT

An apparatus for determining intraocular pressure includes a transducer assembly containing an applanation tonometer for the determination of a cornea applanation pressure and an ultrasonic pachymeter to determine the thickness of the cornea at the site of applanation. The assembly has a tip end which includes an applanation surface and an ultrasonic coupler surface, and an end cap membrane holder is adapted to fit over the tip end of the transducer assembly and hold a thin film membrane stretched over the applanation and ultrasonic coupler surfaces. The transducer assembly also has a detector for detecting the presence or absence of the end cap membrane holder, the detector may generate a signal to disable movement of the transducer assembly if the end cap is not detected.

### REFERENCE TO RELATED APPLICATIONS

This application is a continuation in part of application Ser. No. 10/890,615 filed Jul. 14, 2004, and also claims priority for any new matter to provisional application 60/724,086 filed Oct. 6, 2005. This application further claims priority through the parent application Ser. No. 10/890,615 to a provisional patent application having Ser. No. 60/489,681, which was filed on Jul. 24, 2003.

### FIELD OF THE INVENTION

The invention relates to the field of devices and methods for measuring intraocular pressure for diagnostic and treatment purposes; and to the specific field of devices and methods using ultrasonic pachymetery to calibrate applanation tonometry readings for variations in cornea thickness in order to yield more accurate measurement of intraocular pressure.

### BACKGROUND OF THE INVENTION

Glaucoma refers to a specific pattern of optic nerve damage and visual field loss caused by a number of different eye diseases. Frequently, these diseases are characterized by elevated intraocular pressure; a leading risk factor for development of glaucoma. Devices that measure intraocular pressure are referred to as tonometers.

A particular method of measuring intraocular pressure is known as applanation tonometery, a pressure measurement technique based on the principal that pressure inside a liquid filled sphere can be determined by measuring the force required to flatten a portion of the surface. Applanation tonometers measure either the degree of indentation of the cornea produced by an application probe, or they measure the force required for the probe to flatten a defined area of the cornea, and then translate the measurement into an indication of intraocular pressure. Applanation tonometery was popularized by Goldmann as an improved method of intraocular pressure determination in comparison to indentation tonometery or invasive intraocular pressure measurements. Goldmann applanation tonometery uses and indirect pressure measurement technique based on the Imbert-Fink principal which teaches that pressure inside a liquid filled sphere can be determined by measuring the force required to flatten a portion of the surface. There are several indirect measurement devices in addition to the Goldmann tonometer that have been conceived, e.g. the Mackey Marg, Perkins and Draeger to name a few. They measure either the degree of indentation of the cornea produced by an application probe or they measure the force required for the probe to flatten a defined area of the cornea. Details of such previous devices are widely available in numerous textbooks and will not be discussed herein.

It is known that variations in thickness of the cornea affect the accuracy of applanation pressure techniques. A thinner than normal cornea would flatten more readily than a normal thickness cornea, and generate a falsely low estimate of intraocular pressure. Conversely, a thicker than normal cornea would overestimate the true intraocular pressure.

Recently, studies of ocular hypertensive patients sponsored by the National Eye Institute (NEI) of the National Institutes of Health (NIH) have demonstrated that corneal thickness is the single most important predictor of glaucoma. Corneal thickness is inversely proportional to the risk of developing glaucomatous damage. That is to say, among ocular hypertensives, the thinner the cornea the greater the risk of glaucoma.

Variations in corneal thickness can be measured by optical or ultrasonic means called pachymeters. However, it is time-consuming and expensive to use a second instrument, e.g. an ultrasonic pachymeter, sequentially with the tonometer. Moreover, it is impossible to know if the portion of the cornea applanated for tonometery was the portion whose thickness was measured. Further, the determination of both applanation tonometery and corneal pachymetry requires solving an equation in order to calculate the true intraocular pressure. As a result, the correction of applanation tonometery for corneal thickness variables is generally not widely done except in academic or research circumstances.

During Goldmann applanation tonometery, a fluorescent dye is applied to the corneal surface to aid in the pressure measurement. In an upright patient, the operator looks through the ocular of a slit lamp microscope in order to obtain a clear view of the cornea through the applanation device. Under direct vision and control of the operator, the applanation element is momentarily pressed onto the cornea. The cornea flattens as a result of the force applied by the applanation element. This in turn causes a change in the pattern of fluorescence. The operator observes these changes and when the pattern of fluorescence reaches a predetermined endpoint the intraocular pressure is determined. This method also helps to reduce inadvertent trauma to the delicate epithelial layer of the cornea. This technique, as well as measurements with the classical tonometers, requires training, skill and experience because it is important not to under applanate or over applanate the cornea.

U.S. Pat. No. 6,083,161 and CIP Ser. No. 10/234,294, filed on Sep. 3, 2002, disclose a new apparatus and method which provides more accurate intraocular pressure determination. The apparatus measures conventional tonometery as well as corneal thickness using a single integrated device. Both measurements are made on the exact same region of the cornea. The apparatus uses a transparent corneal applanation element for the determination of the applanation pressure. An ultrasonic transducer is preferably coaxial with or part of the tonometer transducer and is used to measure corneal thickness. Such a design would normally partially skewer the view of the cornea and make the measurement difficult or impossible. However, the apparatus uses an internal reflection technique in order to view around the obscuration. This improved method still suffers, however, from the difficulty of measurement through use of fluorescent dye viewed through a generally non-mobile slit-lamp microscope with patients seated in an upright position. Further, it requires a well-trained and skilled operator in order to obtain accurate and repeatable results.

Hyman teaches a method for determining intraocular pressure using a conventional slit lamp-based Goldmann style tonometer and a pachymeter correcting for corneal thickness. After the pachymeter signal is generated, this method requires the application probe to be moved in a direction toward the subjects' eye until a measurement endpoint is observed by the observer. This method is cumbersome and costly. In addition, the method requires the application probe to be in contact with the cornea for a long time. Contact with the cornea for an extended period of time can alter the intraocular pressure and is uncomfortable for the patient.

There are instances where accurate IOP determination is required and where skilled operators are not present, e.g. examining patients during hospital rounds, emergency rooms, private ophthalmic and optometrist's offices, intern's offices, etc. Further, the use of a portable or handheld tonometer is beneficial or required when the patient is not in an upper right position, e.g. the operating room during surgery, use with children and infants and during patient rounds on the hospital floors. While there are some portable tonometers available, they cannot measure or correct for corneal thickness.

### SUMMARY OF THE INVENTION

There exists a need, therefore, for a simple to use, portable device that does not require trained personnel to simultaneously perform tonometery and pachymetry, that registers more accurate intraocular pressure for general clinical use, and can be used in any patient position. The present invention applanates the cornea with an ultrasonic transducer while simultaneously recording applanation pressure and corneal thickness in the exact region of applanation. The present invention can be configured for use as either a fixed or mobile device and can be used in any position. A microprocessor converts the applanation pressure to an adjusted intraocular pressure, which more accurately reflects the true intraocular pressure when compared to conventional applanation tonometery. This device and method allows for quick, convenient, easy to use, portable and precise determination of intraocular pressure.

The device also may use a transparent membrane that covers the contact tip of the ocular probe, which provides a sterile barrier and prevents tear fluid from the eye from migrating into the probe. The membrane may be stretched over the contact tip by a membrane holder end cap holder cap. The device may have an end cap detection system and an interlock system to prevent the device from operation unless a protective membrane holder is in place.

The shape of the ultrasound transducer crystal may be flat, or more preferably have a curved concave surface that conforms to a convex surface of the acoustic coupler.

The structure of the force coupler between the applanation disc and the force sensor a may be a unitary coupling, or more preferably, a two-segment coupler wherein a small diameter sensor rod with a rounded tip passes through the ultrasonic transducer and contacts a larger surface of a transducer rod.

Other objects, purposes and aspects of this invention will become apparent upon review of the invention as described herein. However, the invention is not intended to be restricted in form nor limited in scope to the embodiments described, but rather is intended to include the full scope of the claims appended hereto.

### BRIEF DESCRIPTION OF THE DRAWINGS

FIG. 1 is an illustration of the present invention showing a tonometery/pachymeter system handpiece in use on a human eye according to the present invention and providing a more accurate intraocular pressure determination.

FIG. 2 is a cross section of a first embodiment of a tonometer/pachymeter handpiece assembly having a pressure measurement means located proximal to the applanation surface and in functional relation to the cornea for determining uncorrected intra-ocular pressure. It is located concentrically within the distal end of the handpiece with an ultrasonic transducer and acoustic coupler for measuring corneal thickness.

FIG. 3 is a partial cross-section of a second embodiment of a tonometer/pachymeter handpiece and transducer assembly having a pressure measurement means in the distal end of the probe subjacent to an ultrasonic transducer assembly showing the ultrasonic transmission and reflection signals for determination of corneal thickness.

FIG. 4A is a partial cross section of a transducer assembly similar to that shown in FIG. 2, highlighting the pressure measurement means which includes a displacement extension rod for transferring force from the cornea to a pressure transducer and with an external pressure coupling membrane covering the cornea contact surface.

FIG. 4B is a preferred in body meant showing a partial cross section of a transducer assembly in accordance with the present invention, highlighting a fluid relaying mechanism for transferring force from the cornea to a pressure transducer.

FIG. 5 is a cross section of the present invention having a displacement transducer for determining uncorrected intraocular pressure;

FIG. 6 is a partial cross-section of an embodiment of the tonometer/pachymeter transducer handpiece assembly having a corneal thickness measuring ultrasonic transducer assembly concentrically located in the distal end of the probe and having a pressure transducer subjacent and centrally positioned therein along with an eye-stabilizing fixation point and pushbutton actuator;

FIG. 7 is another embodiment of the present invention utilizing multiple corneal positioning sensors located within the corneal contact surface area of a tonometer/pachymeter transducer assembly;

FIG. 8 is a typical pressure measurement signal collected in accordance with the present invention; and

FIG. 9 is a typical ultrasound signal for cornea thickness determination collected in accordance with the present invention.

FIG. 10 depicts a relay mechanism to convey the applanation force from the corneal contact surface to the force transducer that includes a displacement coupling that is constructed of two segments.

FIG. 11 is a cross section of a transducer assembly in which the acoustic coupler has a central aperture to pass the displacement sensor rod, and is tapered in shape with a flat top surface that is in contact with the flat surface of the transducer crystal, depicting that only a portion of the acoustic waves can reflect in direct path from the cornea to the crystal.

FIG. 12 is a cross section of an alternative transducer assembly in which the acoustic coupler has a central aperture to pass the displacement sensor rod, and is tapered in shape with a curved convex top surface that is in contact with a concave surface of the transducer crystal, depicting that lager a portion of the acoustic waves can reflect in direct path from the cornea to the crystal.

FIG. 13 is a cross section of an alternative transducer assembly that includes a detector system for detecting that a membrane cover is in proper position over the probe tip.

FIG. 14 shows an end view of the transducer assembly of FIG. 13.

FIG. 15A is a perspective view of an alternative transducer assembly similar to that of FIG. 13.

FIG. 15B is a perspective view of the transducer assembly of FIG. 15A shown with the cap partially removed.

FIG. 15C is a perspective view of the transducer assembly of FIG. 15A shown without the cap.

FIG. 16 is a cross section of an alternative transducer assembly having an alternative detection system.

### DESCRIPTION OF PREFERRED EMBODIMENTS

It is a preferred embodiment of the present invention to obtain more accurate intraocular pressure measurements using a solid-state, ultrasonic cornea thickness measuring means working in the 10 to 20 MHz frequency domain in functional association with a pressure sensing means as an applanation surface of predetermined area for contact with the corneal surface.

In another preferred embodiment, the applanation surface is a replaceable membrane.

In another preferred embodiment, the pressure sensing means is located proximal to the applanation surface and in functional relation to the corneal surface.

In another preferred embodiment, the device displays a digital LED readout of the applanation pressure, the corneal thickness and the intraocular pressure adjusted for corneal thickness.

It is yet a further preferred embodiment in which the measurement system incorporates a sensing means responsive to proper positioning of the system.

### Example 1

A patient preparing for Laser Assisted In situ Keratomileusis (LASIK) photorefractive surgery for minus eight diopters (−8 D) of myopia has a preoperative central corneal thickness of 452 microns. Six months following the LASIK procedure the intraocular pressure is measured as determined by Goldmann tonometery as 16 mmHg. The uncorrected intraocular pressure as determined by the present invention is also 16 mmHg. Pachymetry indicates the central corneal thickness to be 347 microns. The corrected intraocular pressure as determined by the present invention is 25 mmHg. In this example the present invention demonstrated that the intraocular pressure was higher than would be otherwise apparent; potentially masking glaucoma. The normal intraocular pressure ranges from 12 to 21 mmHg.

### Example 2

A patient presented for a routine of found that examination has an intraocular pressure of 19 mmHg as determined by Goldmann tonometery. The uncorrected intraocular as determined by the present invention is also 19 mmHg. Pachymetry indicates the central corneal thickness to be 485 microns. The corrected intraocular pressure as determined by the present invention is 23 mmHg. In this example the present invention demonstrated that the intraocular pressure was higher than would be otherwise apparent; masking glaucoma.

The apparatus of this invention described and shown herein is a novel device for simultaneous measurement, at the same locus of applanation, pressure and surface thickness of a fluid filled sphere for more accurate determination of intracavity pressure, wherein at least a portion of the applanation surface is an ultrasonic transducer. The method for utilizing this device includes the simultaneous measurement, at the same locus of applanation, intracavity pressure and surface thickness of a fluid filled sphere for more accurate determination of intracavity pressure. In addition this novel device provides for simultaneous measurement, at the same locus of applanation, tonometery and pachymetry for determination of more accurate intraocular pressure, wherein at least a portion of the applanation surface is an ultrasonic transducer. Further, the method and device of the invention herein can provide for a fixation light source to stabilize the patient eye during applanation. Further yet, this invention includes a method of simultaneous measurement, at the same locus of applanation tonometery and pachymetry for the purpose of more accurate intraocular pressure determination. The locus of applanation tonometery and pachymetry is preferably the cornea of the eye.

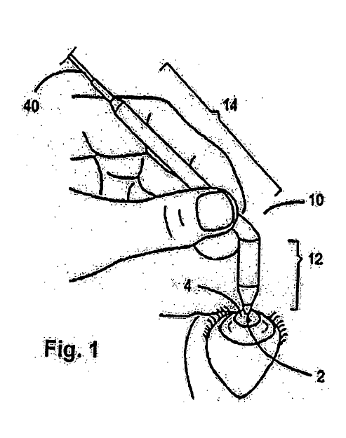

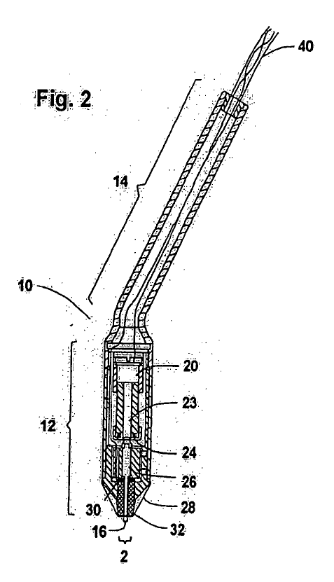

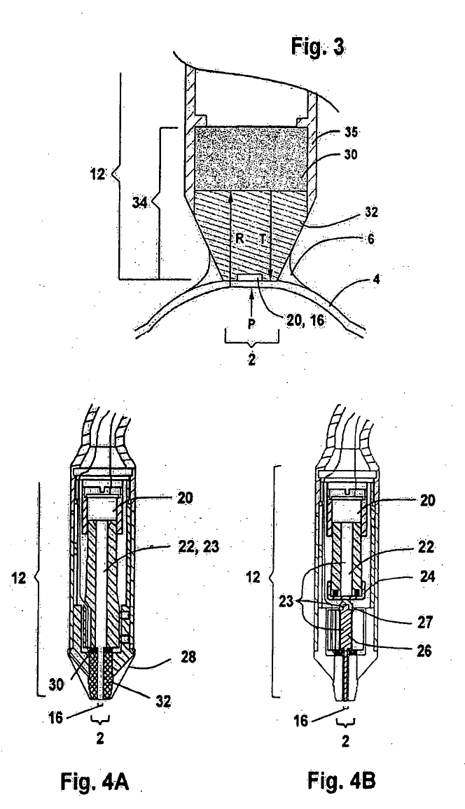

Referring now to the drawings, FIG. 1 illustrates a tonometer/pachymeter handpiece 10 suitable for contact by corneal contact surface 2 to cornea 4 and containing transducer assembly 12 and handpiece wand 14 according to an embodiment of the present invention. Tonometer/pachymeter transducer assembly 12 as shown in greater detail in FIG. 2 and FIG. 3 includes ultrasonic transducer assembly 33 and pressure transducer 20.

As illustrated in FIG. 3, ultrasonic transducer assembly 33 includes an ultrasonic transducer crystal 30 and an acoustic coupler 32, which can be made of any material suitable to transmit ultrasonic waves. Ultrasonic waves T are generated from ultrasonic transducer crystal 30 and transmitted or intensified through acoustic coupler 32. Ultrasonic waves R return to crystal 30 through acoustic coupler 32 following reflection or echo from distal surface of cornea 4. Ultrasonic transducer assembly 33 is held in position by outer housing 35. Force from cornea 4 is sensed by pressure transducer 20.

As also shown in FIG. 3, the pressure force transducer 20 may be embedded in and subjacent to the acoustic coupler 32 in the distal end of assembly 12 and make up a portion of cornea contact surface 2. When the cornea contact surface 2 of the transducer assembly 12 is gently pressed or applanated and momentarily flattens the cornea 4 to an area beyond the pressure sensitive area 16, the only force sensed will be caused by the intraocular pressure. If the pressure sensitive area 16 is 3.06 mm in diameter, the measured IOP is the same as that from a Goldmann instrument without orbit furry corrections. It should be noted that while any size pressure sensitive area 16 can be used, the smaller the surface area the least trauma for the patient.

As shown in FIG. 4A and FIG. 4B, a pressure transducer 20 may be located in the transducer assembly apart from the cornea contact surface 2, and a relay mechanism 23 may be used to transfer pressure from the cornea surface 2 to the pressure transducer 20. The relay mechanism 23 may be a chamber containing air or another fluid 22 as shown in FIG. 4A or be a solid material. The relay mechanism 23 may also be comprised of multiple parts, such as a displacement extension rod 26, a coupler 27 and a fluid 22 as shown in FIG. 4B. Alternatively, the relay mechanism 23 may be a displacement extension rod 26 coupled directly to pressure transducer 20. The relay mechanism 23 may be is air or other gaseous fluid, sealed to the environment through an external pressure coupling membrane 28. The external pressure coupling membrane 28 can also serve as a sterile barrier for contact with the cornea 4., and can also be used to seal the relay mechanism 23.

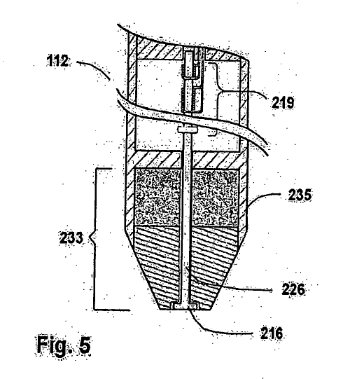

Alternatively, as shown in FIG. 5, determination of IOP can be accomplished by use of displacement transducer 219 and displacement extension rod 226 that will generate a signal proportional to the indentation of pressure sensitive area 216. Cornea contact surface 2 creates an ultrasonic junction with cornea 4 that transmits ultrasonic transducer crystal 30 signals to and communicates reflected ultrasound signals from cornea 4. The ultrasonic signal reflected from the posterior surface of cornea 4 and communicated back through acoustic coupler 32 and detected by ultrasonic transducer crystal 30 is proportional to the thickness of the cornea. Transducer assembly 12 is preferably positioned at the geometric center of corneal cornea 4. Signal conditioning electronics and microprocessor (not shown) are programmed to receive output signals from ultrasonic transducer crystal 30 and pressure transducer 20 and display intraocular pressure measurements corrected for corneal thickness; the true intra-cavity pressure.

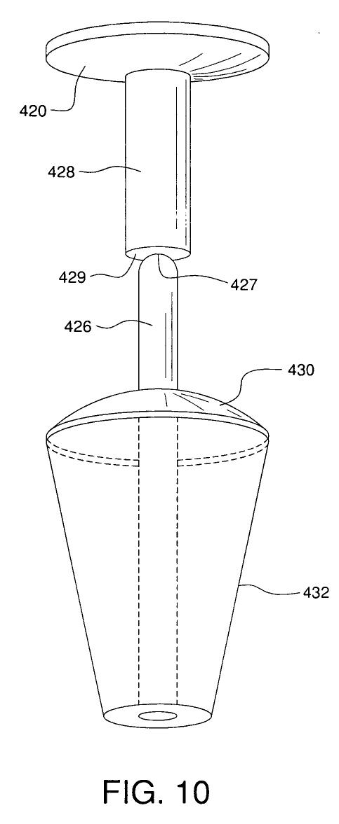

Alternatively, as shown in FIG. 10, the relay mechanism to convey the applanation force from the corneal contact surface 2 to the force transducer 420 may include a displacement coupling that is constructed of two segments. The first segment is a small diameter sensor rod 426 passing through the ultrasonic transducer 430 and accoustic coupler 432. The other segment is a larger diameter force transducer rod 428 that is coupled to the force transducer 430. The sensor rod has a sharply rounded tip 427 that produces a small contact area with the flat surface 429 of the larger transducer rod. This arrangement reduces friction force from the sonic coupler and holding mechanism to less than would be caused a larger diameter unitary rod coupling, and alleviates the bending effect of the compressive force on the thin sensor rod.

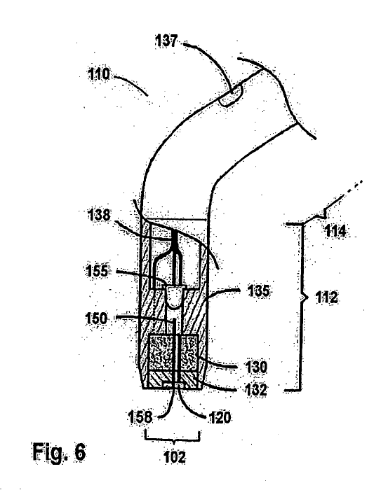

FIG. 6 illustrates another embodiment of the interior elements of a tonometer/pachymeter handpiece 110 in accordance with the present invention. In this configuration, the contact surface 102 is formed from the tapered distal portion of the outer jacket 135, acoustic coupler 132, pressure transducer 120 and a fixation point 158. Fixation point 158 is shown as the distal end of the optical coupler 150. The optical coupler 150 is shown as a short length of fiber optic but can be any other optical transmitting material or air. It is illuminated by a light emitting diode 155 or similarly functional illuminating device.

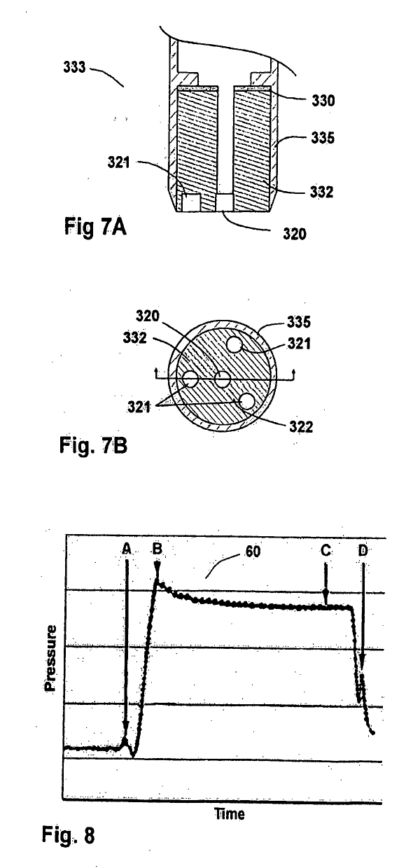

FIG. 7A and FIG. 7B show a cross-section and end view, respectively, of an ultrasonic transducer assembly 333 consistent with the teaching of the invention in which multiple cornea positioning transducers 321 are shown. In the illustrated embodiment, three cornea positioning transducers 321 are concentrically located 120° around pressure transducer 20. However, the positioning transducers 321 can be positioned any distal location provided they are selected to be responsive to contact with the cornea 4. In this configuration, signals can be produced indicating that cornea contact surface 2 is uniformly and perpendicularly in contact with cornea 4.

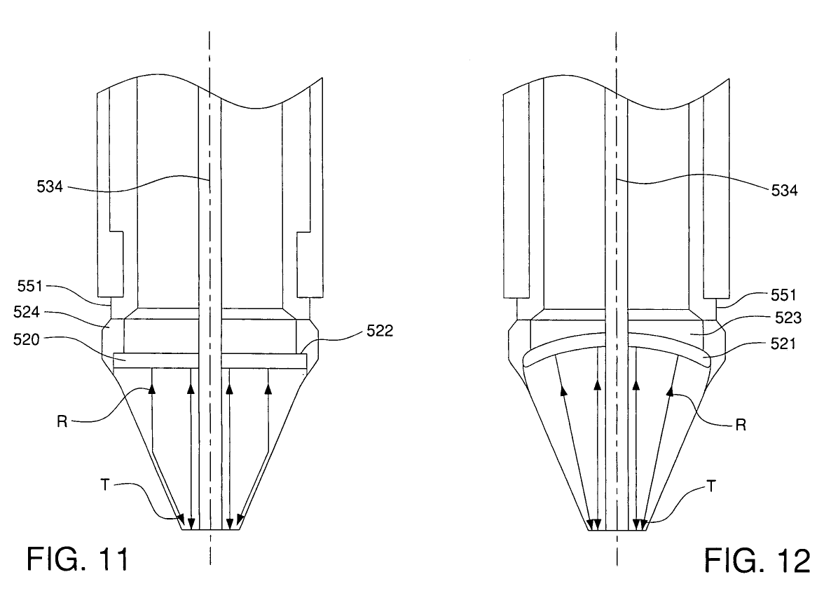

As shown earlier in FIG. 3, the ultra sonic transmission and reflection path are depicted as direct path rays T and R when using a flat surface transducer crystal 30 and an acoustic coupler 532. As shown in FIG. 11, when the acoustic coupler 532 has a central aperture to pass the displacement sensor rod, and is tapered in shape with a flat top surface 522 that is in contact with the corresponding flat surface of the transducer crystal 524, only a portion of the acoustic waves T and R can reflect in direct path from the cornea to the crystal. Other portions of the waves are refracted along the sides of the coupler 532.

FIG. 12 shows an improved transducer assembly in which the transducer crystal 523 and the acoustic coupler 521 have conforming curved surfaces, such that the crystal 523 has a concave surface and the upper surface of the coupler 521 is correspondingly convex. As shown by the ray paths T and R in these figures, a greater portion of the acoustic waves impinge on the cornea with less refracted off of the sides of the coupler.

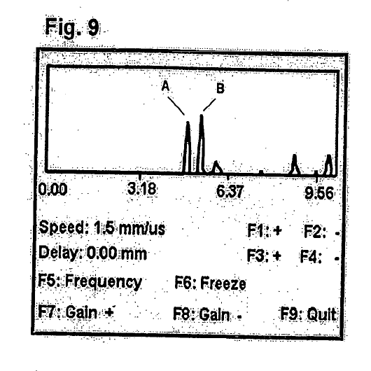

FIG. 8 depicts data representative of a typical pressure measurement signal generated using the configuration shown in FIG. 4A where pressure signal 60 is a pressure versus time tracing of pressure exerted on pressure transducer 20 resulting from applanation of cornea contact surface 2 on cornea 4. Pressure signal 60 at time ‘A’ represents initial depression of cornea contact surface 2 to cornea 4. ‘B’ represents a signal overshoot, ‘C’ represents true applanation pressure not corrected for thickness of cornea 4 and ‘D’ represents buckling of cornea 4 resulting from excessive force on cornea contact surface 2. Signal conditioning electronics (not shown) assess the data representative of pressure measurements and extracts and display true intraocular pressure ‘C’.

FIG. 9 depicts data representative of ultrasonic waves generated by ultrasonic transducer crystal 30 and reflecting from cornea contact surface 2 (signal ‘T’ in FIG. 3) and shown as peak intensity ‘A’ and ultrasonic waves reflecting from the distal surface of cornea 4 (signal ‘R’ in FIG. 3) and shown as peak intensity ‘B’. Time difference between peak intensity ‘A’ and ‘B’ is proportional to thickness of cornea 4.

In the embodiment shown in FIG. 4A, a removable transparent membrane 28 may be placed over the portion of the probe tip that comes into contact with the surface of the cornea. The membrane forms a sterile barrier that prevents eye liquids from entering the interior of the probe, where the liquid would interfere with the fine calibration of the probe. The removable membrane is also intended to be a single use disposable piece to prevent transmission of microorganisms from patient to patient.

With the need to remove a contaminated membrane after each use, there is a substantial risk of forgetting to replace the membrane before the next use. Given the serious effect of an unprotect use on the delicate instrument, it would be highly beneficial to have some type of lockout protection that inhibits the device from operating unless a membrane is in place, and in a manner that prevents or warns against the movement of the probe toward contact with the patient's eye. Thus, as shown in shown in FIGS. 13, 14 and 15, a transducer assembly 610 includes a detector system for detecting that a membrane cover is in proper position over the probe tip. The detector system includes a light source 656, am optical fiber light pipe 657, a removable membrane holder or end cap 642, a return optical fiber light pipe 659 and a light sensor 652. The membrane holder holds the thin membrane 644 taut over the tip of the probe. The holder or end cap 642 has an upper edge 661 that is formed to resiliently snap into an annular groove fitting 662 on the probe, The light pipe 657 terminates in the groove fitting such that light emanating from the pipe) impinges on the portion of the holder that is near the pipe in the groove fitting, and the return light pipe 659 terminates in the groove fitting on the opposite side from the first pipe. The holder itself is constructed of a light transmissive material, such that light from the light source can pass from the first light pipe through the holder and into the second light pipe, where it is transmitted to and detected by the light detector 652. If no holder is in place, light from the source cannot enter the second pipe and cannot be detected by the light detector. Thus, the detection of light by the detector can be used as a signal that a membrane is in place, and as a switch allowing operation of the probe. There are various known ways in which a light sensing switch can be used to prevent or enable operation or movement of the device depending upon a signal indicating the presence or absence of the membrane holder, or the light signal can be transformed into a visual or other auditory warning.

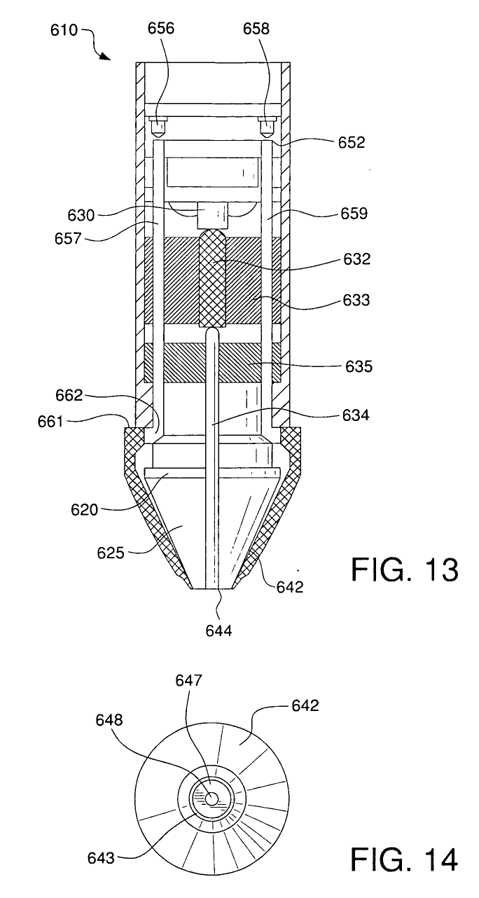

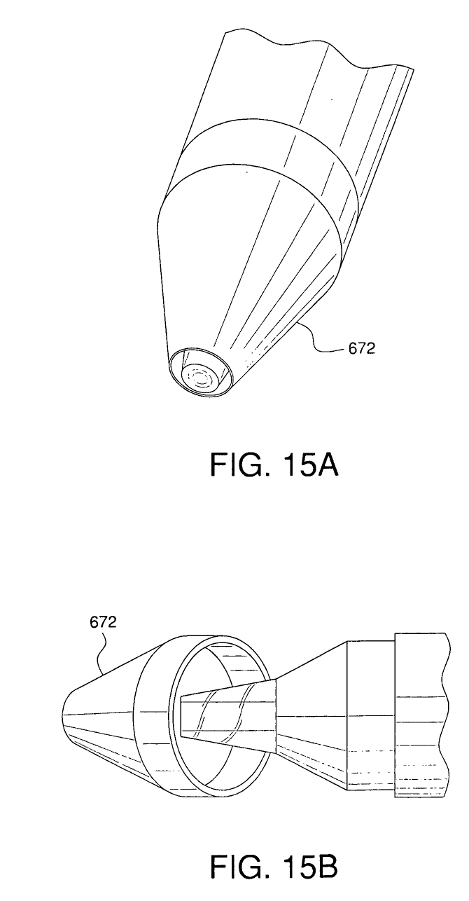

Another advantage of this light transmitting membrane holder 642 is that it can be shaped as in FIG. 13 to taper toward the tip end and terminate near the tip end. Since it will transmit light to the tapered end, if the light is a monochrome, such as from a red LED source, the tapered end will appear as a small monochrome circle 643 to the patent, as shown in FIG. 14. This circle provides a convenient fixation target for the patient to focus to as the probe is brought into eye contact.

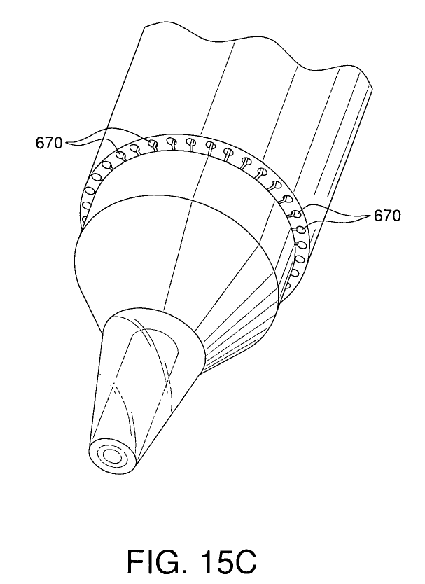

FIGS. 15A-15C show another alternate form of light sensing interlock using distributed optical fiber channels 670 to detect the presence or absence of a membrane holder end cap 672. The membrane holder cap is partly opaque and partly reflective. Light reflected from the membrane cap 672 returns to a light sensing detector. Light transmitted through the cap produces a circular fixation target for the patient.

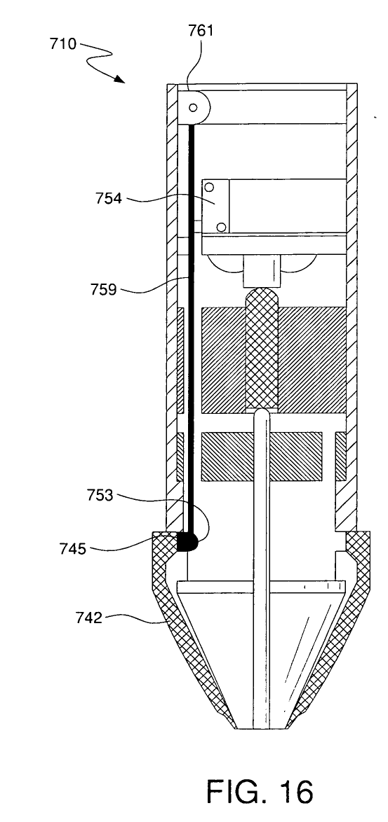

FIG. 16 shows an alternate form of membrane cover interlock 710. In this embodiment, a simple feeler lever 759 terminates in the groove fitting 753. When a membrane cover 742 is in place, the upper edge of the cover displaces the feeler inward. This movement is sensed at the opposite end of the feeler by a mechanical electrical transducer 761 or switch that transforms the feeler movement into an electrical signal that can be used as an indication that a membrane is in place and as a switch allowing operation of the probe.

Variations or modifications to the subject matter of this invention may occur to those skilled in the art upon review of the summary provided herein, in addition to the description of its preferred embodiment, in light of the drawings. Such variations, if within the spirit of this invention, are intended to be encompassed within the scope of the invention as described herein.

We claim:

1. An apparatus for determining intraocular pressure comprising:
a transducer assembly containing an applanation tonometer for the determination of a cornea applanation pressure and an ultrasonic pachymeter to determine the thickness of the cornea at the site of applanation, the assembly having a tip end which includes an applanation surface and an ultrasonic coupler surface;
an end cap membrane holder adapted to fit over the tip end of the transducer assembly and hold a thin film membrane stretched over the applanation and ultrasonic coupler surfaces; and
a detector for detecting the presence or absence of the end cap membrane holder.

2. An apparatus as in claim 1, wherein the detector includes a light source and a light sensor, and an optical pathway from the source to the sensor that passes through a portion of the end cap.

3. An apparatus as in claim 1 wherein the detector includes a light source and a light sensor, and an optical pathway from the source to the end cap and a second optical pathway that conducts light reflected off of the end cap to the sensor.

4. An apparatus as in claim 2 wherein some of the light from the light source is transmitted through the end cap holder and appears to a patient under examination as target circle surrounding the applanation and ultrasonic coupler surfaces.

5. An apparatus as in claim 3 wherein some of the light from the light source is transmitted through the end cap holder and appears to a patient under examination as target circle surrounding the applanation and ultrasonic coupler surfaces.

6. An apparatus as in claim 1, wherein the detector includes a mechanical feeler that is displaced when the end cap is placed over the tip end of the transducer assembly having a first end that is displaced when

7. An apparatus as in claim 6, further comprising the displacement of the mechanical feeler causing the closing or opening of an electromagnetic circuit.

8. An apparatus as in claim 1, further comprising the detector generating a signal that can be used to enable or disable movement of the transducer assembly.

9. An apparatus as in claim 1, further including a displacement coupling to transfer the force of applanation to a pressure transducer, the coupling being comprised of a small diameter sensor rod extending from the tip end into the transducer assembly to contact an input surface of the pressure transducer.

10. An apparatus as in claim 9, wherein the sensor rod has a rounded tip that contacts the input surface of the pressure transducer.

11. An apparatus as in claim 1, wherein the ultrasonic pachymeter includes a transducer crystal and an acoustic coupler, and the crystal and coupler have conforming curved mating surfaces.

12. An apparatus as in claim 11, wherein the crystal has a concave surface and the corresponding mating surface of the coupler is convex.
